(19) United States
(12) Patent Application Publication
(10) Pub. No.: US 2026/0048238 A1
(43) Pub. Date: Feb. 19, 2026

(54) HANDLE FOR A STEERABLE MEDICAL DEVICE HAVING DUAL STEERING KNOBS

(71) Applicant: Boston Scientific Scimed, Inc., Maple Grove, MN (US)

(72) Inventor: Jeffery D. ARNETT, Louisa, VA (US)

(21) Appl. No.: 19/265,473

(22) Filed: Jul. 10, 2025

Related U.S. Application Data
(60) Provisional application No. 63/683,086, filed on Aug. 14, 2024.

(51) Int. Cl. A61M 25/01

(52) U.S. Cl. CPC A61M 25/0147; A61M 25/0136; A61M 2025/015

(57) ABSTRACT

A handle for a steerable medical device includes a grip having a distal end and a proximal end. A spindle extends from the distal end of the grip. The spindle includes parallel first and second walls. A planar inner surface of the first wall and the second wall defining a guide channel. A proximal wire holder is translatable along the guide channel. A proximal knob is rotatably coupled to the spindle for moving the proximal wire holder in a proximal direction to manipulate a first control wire of a plurality of control wires to produce a change in a deflection of the medical device in a first direction. The proximal knob is configured for moving the proximal wire holder in a distal direction to manipulate a second control wire to produce a change in a deflection of the medical device in a second direction opposite the first direction.

### CROSS REFERENCE TO RELATED APPLICATIONS

This application claims priority to U.S. Provisional Patent Application No. 63/683,086 entitled “HANDLE FOR A STEERABLE MEDICAL DEVICE HAVING DUAL STEERING KNOBS,” filed Aug. 14, 2024, which is hereby incorporated by reference in its entirety.

### TECHNICAL FIELD

The disclosure relates to a handle for a medical device. More specifically the disclosure relates to a handle for a medical device that enables steering of the medical device in a body of a patient.

### BACKGROUND

U.S. Pat. No. 5,944,690 granted to Falwell et al. discloses a steerable catheter control mechanism for manipulating a pair of control wires which utilizes a slider mechanism coupled to the proximal ends of the control wires. However, the slider mechanism disclosed by Falwell lacks ease of use as it is awkward to grasp and use. Furthermore, the disclosed slider mechanism provides limited control in steering the catheter. The device provides a thumb control that lacks precision. It is unable to provide precise steering of the catheter as it lacks resolution for permitting minute manipulations needed to provide slight changes in the deflection of the catheter.

U.S. Pat. No. 7,691,095 granted to Bednarek et al. discloses a bi-directional steerable catheter control handle which includes an adjustment knob rotatably connected to the handle. Rotation of the handle results in deflection of two sliding members (each connected to a pull wire) in opposite directions, resulting in respective deflection of the distal end of the catheter. However, the steerable control handle provided by Bednarek is complex and difficult to manufacture.

### SUMMARY

Example 1 is a handle for a steerable medical device. The medical device includes a plurality of control wires, a distal end of the plurality of control wires being coupled to the medical device at a distal region thereof. The handle includes a grip having a distal end and a proximal end. A spindle extends from the distal end of the grip. The spindle includes a first wall and a second wall parallel to the first wall. A planar inner surface of the first wall and the second wall defining a guide channel. A proximal wire holder is translatable along the guide channel. A proximal knob is rotatably coupled to the spindle for moving the proximal wire holder in a proximal direction to manipulate a first control wire of the plurality of control wires to produce a change in a deflection of the medical device in a first direction. The proximal knob is also configured for moving the proximal wire holder in a distal direction to manipulate a second control wire of the plurality of control wires to produce a change in a deflection of the medical device in a second direction opposite the first direction.

Example 2 is the handle of Example 1, further comprising a distal wire holder translatable along the guide channel.

Example 3 is the handle of Example 2, further comprising a distal knob rotatably coupled to the spindle for moving the distal wire holder in a proximal direction to manipulate a third control wire of the plurality of control wires to produce a change in a deflection of the medical device in a third direction. The distal knob is also configured for moving the distal wire holder in a distal direction to manipulate a fourth control wire of the plurality of control wires to produce a change in a deflection of the medical device in a fourth direction opposite the third direction.

Example 4 is the handle of any of Examples 1-3, further comprising a nose cap, wherein the spindle includes a distal end having a groove for receiving a flange from a medical device and the nose cap is configured to secure the flange in the groove.

Example 5 is the handle of any of Examples 1-4, wherein the first wall and the second wall each include a curved outer surface, and a left and right planar surface orthogonal to the planar inner surface.

Example 6 is the handle of any of Examples 2 - 5, wherein the spindle comprises a divider wall within the guide channel separating the guide channel into a proximal guide channel and a distal guide channel, and the proximal wire holder is limited to movement within the proximal guide channel and the distal wire holder is limited to movement within the distal guide channel.

Example 7 is the handle of any of Examples 1-6, wherein the grip is hollow and comprises a right half and a left half connected by one or more connector.

Example 8 is the handle of Example 7, wherein the grip houses a first pulley, second pulley, and a guide funnel.

Example 9 is the handle of Example 8, wherein the first pulley is located distal of the second pulley, the guide funnel is located distal of the first pulley, and the second pulley has a radius larger than a radius of the first pulley.

Example 10 is the handle of any of Examples 3-9, wherein the proximal knob and the distal knob include an outer portion surrounding a cylindrical inner portion, the cylindrical inner portion including internal threads.

Example 11 is the handle of Example 10, wherein the inner portion comprises a right half and a left half.

Example 12 is the handle of any of Examples 10 or 11, wherein the proximal wire holder includes curved threaded portions configured to mate with the internal threads of the cylindrical inner portion of the proximal knob, and the distal wire holder includes curved threaded portions configured to mate with the internal threads of the cylindrical inner portion of the distal knob.

Example 13 is the handle of any of Examples 1-12, wherein the proximal wire holder comprises a first piece and a second piece. The first piece and the second piece are configured to join together to create an opening and to secure the first control wire in a first notch and the second control wire in a second notch. The first control wire includes a crimp configured to contact a proximal surface of the proximal wire holder when the proximal wire holder is translated in a proximal direction along the guide channel. The second control wire includes a crimp configured to contact a distal surface of the proximal wire holder when the proximal wire holder is translated in a distal direction along the guide channel.

Example 14 is the handle of Examples 3-13, wherein the distal wire holder comprises a third piece and a fourth piece. The third piece and the fourth piece are configured to join together to create an opening and to secure the third control wire in a third notch and the fourth control wire in a fourth notch. The third control wire includes a crimp configured to contact a proximal surface of the distal wire holder when the distal wire holder is translated in a proximal direction along the guide channel. The fourth control wire including a crimp configured to contact a distal surface of the distal wire holder when the distal wire holder is translated in a distal direction along the guide channel.

Example 15 is the handle of any of Examples 1-14, wherein the proximal knob includes a proximal indicator rib, the distal knob includes a distal indicator rib, and the grip includes a grip indicator rib, such that alignment of the proximal indicator rib, the distal indicator rib, and the grip indicator rib indicates a neutral position of the medical device.

Example 16 is a handle for a steerable medical device. The medical device includes a plurality of control wires, and a distal end of the plurality of control wires is coupled to the medical device at a distal region thereof. The handle includes a grip having a distal end and a proximal end. A spindle extends from the distal end of the grip. The spindle includes a first wall and a second wall parallel to the first wall. A planar inner surface of the first wall and the second wall defines a guide channel. A proximal wire holder is translatable along the guide channel. A distal wire holder is translatable along the guide channel. A proximal knob is rotatably coupled to the spindle for moving the proximal wire holder in a proximal direction to manipulate a first control wire to produce a change in a deflection of the medical device in a first direction. The proximal knob is also configured for moving the proximal wire holder in a distal direction to manipulate a second control wire to produce a change in a deflection of the medical device in a second direction opposite the first direction. A distal knob is rotatably coupled to the spindle for moving the distal wire holder in a proximal direction to manipulate a third control wire to produce a change in a deflection of the medical device in a third direction. The distal knob is also configured for moving the distal wire holder in a distal direction to manipulate a fourth control wire to produce a change in a deflection of the medical device in a fourth direction opposite the third direction.

Example 17 is the handle of Example 16, further comprising a nose cap, wherein the spindle includes a distal end having a groove for receiving a flange from a medical device and the nose cap is configured to secure the flange in the groove.

Example 18 is the handle of Example 16, wherein the first wall and the second wall each include a curved outer surface, and a left and right planar surface orthogonal to the planar inner surface.

Example 19 is the handle of Example 16, wherein the spindle comprises a divider wall within the guide channel separating the guide channel into a proximal guide channel and a distal guide channel, and the proximal wire holder is limited to movement within the proximal guide channel and the distal wire holder is limited to movement within the distal guide channel.

Example 20 is the handle of Example 16, wherein the grip is hollow and comprises a right half and a left half connected by one or more connector.

Example 21 is the handle of Example 20, wherein the grip houses a first pulley, second pulley, and a guide funnel.

Example 22 is the handle of Example 21, wherein the first pulley is located distal of the second pulley, the guide funnel is located distal of the first pulley, and the second pulley has a radius larger than a radius of the first pulley.

Example 23 is the handle of Example 16, wherein the proximal knob and the distal knob include an outer portion surrounding a cylindrical inner portion, the cylindrical inner portion including internal threads.

Example 24 is the handle of Example 23, wherein the inner portion comprises a right half and a left half.

Example 25 is the handle of Example 23, wherein the proximal wire holder includes curved threaded portions configured to mate with the internal threads of the cylindrical inner portion of the proximal knob, and the distal wire holder includes curved threaded portions configured to mate with the internal threads of the cylindrical inner portion of the distal knob.

Example 26 is the handle of Example 16, wherein the proximal wire holder comprises a first piece and a second piece. The first piece and the second piece are configured to join together to create an opening and to secure the first control wire in a first notch and the second control wire in a second notch. The first control wire includes a crimp configured to contact a proximal surface of the proximal wire holder when the proximal wire holder is translated in a proximal direction along the guide channel. The second control wire includes a crimp configured to contact a distal surface of the proximal wire holder when the proximal wire holder is translated in a distal direction along the guide channel.

Example 27 is the handle of Example 26, wherein the distal wire holder comprises a third piece and a fourth piece. The third piece and the fourth piece are configured to join together to create an opening and to secure the third control wire in a third notch and the fourth control wire in a fourth notch. The third control wire includes a crimp configured to contact a proximal surface of the distal wire holder when the distal wire holder is translated in a proximal direction along the guide channel. The fourth control wire includes a crimp configured to contact a distal surface of the distal wire holder when the distal wire holder is translated in a distal direction along the guide channel.

Example 28 is the handle of Example 16, wherein the proximal knob includes a proximal indicator rib, the distal knob includes a distal indicator rib, and the grip includes a grip indicator rib, such that alignment of the proximal indicator rib, the distal indicator rib, and the grip indicator rib indicates a neutral position of the medical device.

Example 29 is a handle for a steerable medical device. The medical device includes a plurality of control wires. A distal end of the plurality of control wires is coupled to the medical device at a distal region thereof. The handle includes a grip having a distal end and a proximal end. A spindle extends from the distal end of the grip. The spindle includes a first wall and a second wall parallel to the first wall, a planar inner surface of the first wall and the second wall defines a guide channel. A proximal wire holder is translatable along the guide channel. The proximal wire holder includes a first piece and a second piece. The first piece and the second piece include curved threaded portions. The first piece and the second piece are configured to join together to create an opening and to secure a first control wire of the plurality of control wires in a first notch and the second control wire of the plurality of control wires in a second notch. The first control wire includes a crimp configured to contact a proximal surface of the proximal wire holder when the proximal wire holder is translated in a proximal direction along the guide channel. The second control wire includes a crimp configured to contact a distal surface of the proximal wire holder when the proximal wire holder is translated in a distal direction along the guide channel.

Example 30 is the handle of Example 29, further comprising a proximal knob rotatably coupled to the spindle for moving the proximal wire holder in the proximal direction to manipulate the first control wire to produce a change in a deflection of the medical device in a first direction. The proximal knob is also configured for moving the proximal wire holder in the distal direction to manipulate the second control wire to produce a change in a deflection of the medical device in a second direction opposite the first direction.

Example 31 is a handle for a steerable medical device. The medical device includes a plurality of control wires, a distal end of the plurality of control wires are coupled to the medical device at a distal region thereof. The handle includes a grip having a distal end and a proximal end. A spindle extends from the distal end of the grip. The spindle includes a first wall and a second wall parallel to the first wall. A planar inner surface of the first wall and the second wall defines a guide channel. A divider wall within the guide channel separates the guide channel into a proximal guide channel and a distal guide channel. A proximal wire holder is translatable along the proximal guide channel. A proximal knob is rotatably coupled to the spindle for moving the proximal wire holder in a proximal direction to manipulate a first control wire of the plurality of control wires to produce a change in a deflection of the medical device in a first direction. The proximal knob is also configured for moving the proximal wire holder in a distal direction to manipulate a second control wire of the plurality of control wires to produce a change in a deflection of the medical device in a second direction opposite the first direction.

Example 32 is the handle of Example 31, further including a distal wire holder translatable along the distal guide channel. A distal knob is rotatably coupled to the spindle for moving the distal wire holder in a proximal direction to manipulate a third control wire of the plurality of control wires to produce a change in a deflection of the medical device in a third direction. The distal knob is also configured for moving the distal wire holder in a distal direction to manipulate a fourth control wire of the plurality of control wires to produce a change in a deflection of the medical device in a fourth direction opposite the third direction.

Example 33 is the handle of Example 31, further comprising a nose cap, wherein the spindle includes a distal end having a groove for receiving a flange from a medical device and the nose cap is configured to secure the flange in the groove.

Example 34 is the handle of Example 32, wherein the proximal knob and the distal knob include an outer portion surrounding a cylindrical inner portion, the cylindrical inner portion including internal threads.

Example 35 is the handle of Example 33, wherein the proximal wire holder includes curved threaded portions configured to mate with the internal threads of the cylindrical inner portion of the proximal knob, and the distal wire holder includes curved threaded portions configured to mate with the internal threads of the cylindrical inner portion of the distal knob.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a perspective view of a handle assembly, in accordance with an embodiment the present disclosure.

FIG. 2 in an exploded view of the handle assembly of FIG. 1, in accordance with an embodiment of the present disclosure.

FIG. 3 is a cross-sectional view of the handle assembly along line A-A of FIG. 1, in accordance with an embodiment of the present disclosure.

FIG. 4 is a cross-sectional view along line B-B of FIG. 3, in accordance with an embodiment of the present disclosure.

FIG. 5A is a cross-sectional view of the handle assembly along line A-A of FIG. 1 showing a distal wire holder in a most distal position and a proximal wire holder in a most proximal position, in accordance with an embodiment of the present disclosure.

FIG. 5B is a cross-sectional view of the handle assembly along line A-A of FIG. 1 showing the distal wire holder in a most proximal position and the proximal wire holder in a most distal position, in accordance with an embodiment of the present disclosure

FIG. 6 is a perspective view of a wire holder of the handle assembly of FIG. 1, in accordance with an embodiment of the present disclosure.

FIG. 7 is a perspective view of the distal wire holder and the proximal wire holder over a spindle of the handle assembly of FIG. 1, in accordance with an embodiment of the present disclosure.

FIG. 8A is a first perspective view of a first pulley, a second pulley, and a guide funnel of the handle assembly of FIG. 1, in accordance with an embodiment of the present disclosure.

FIG. 8B is a second perspective view of a first pulley, a second pulley, and a guide funnel of the handle assembly of FIG. 1, in accordance with an embodiment of the present disclosure.

FIG. 9 is a partial cross-sectional view along line C-C of FIG. 1 showing ribs for locating control wires against the first pulley and the second pulley, in accordance with an embodiment of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

Steerable medical devices have various uses and applications, such as for guiding and positioning devices such as catheters, dilators, guidewires, sheaths and the like within a patient's body. Handles used with such steerable devices typically include a mechanism for actuating one or more pull wires capable of deflecting the steerable device and thus steering or guiding a functional tip of a medical device positioned therein.

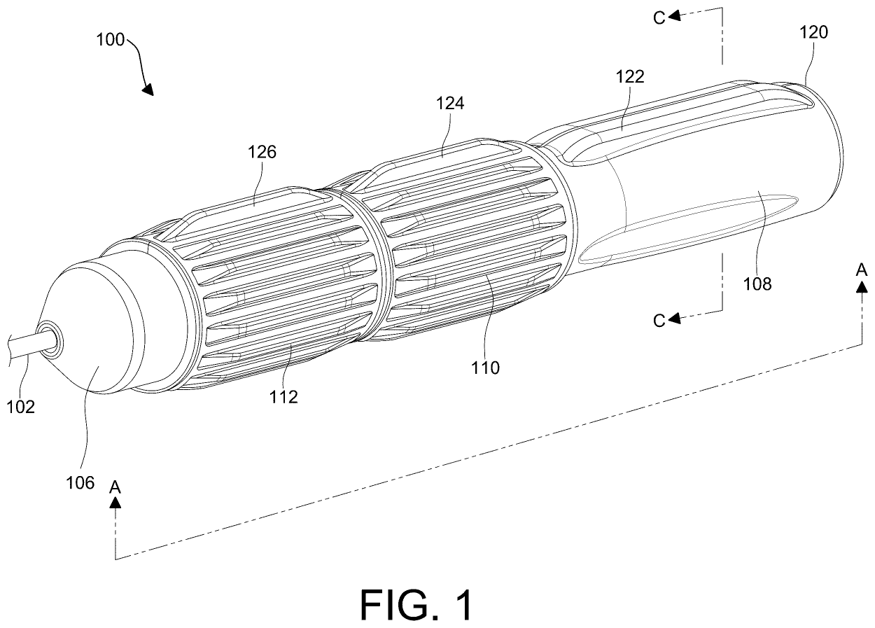

In one embodiment of the present disclosure as shown in FIG. 1, a steerable control assembly or handle 100 is provided for manipulating a medical device 102. The medical device 102 may include, without limitation, a catheter, sheath, dilator, introducer, guidewire, stylet, or similar medical devices. The medical device 102 includes a plurality of control wires for producing a change in deflection of the medical device 102 to allow the medical device 102 to achieve a desired shape. A distal end of each of the plurality of control wires is coupled to the medical device 102 at a distal region thereof such that a tension applied to a proximal end of one of the control wire results in a bend in the medical device 102. The plurality of control wires extend proximally from the connection through one or more channels or lumens to the handle 100. In some embodiments, the medical device 102 may take the form of a tubular member with one or more lumens. In other embodiments, the medical device may include a substantially solid cross-section except for any lumens or grooves configured for holding one or more control wire.

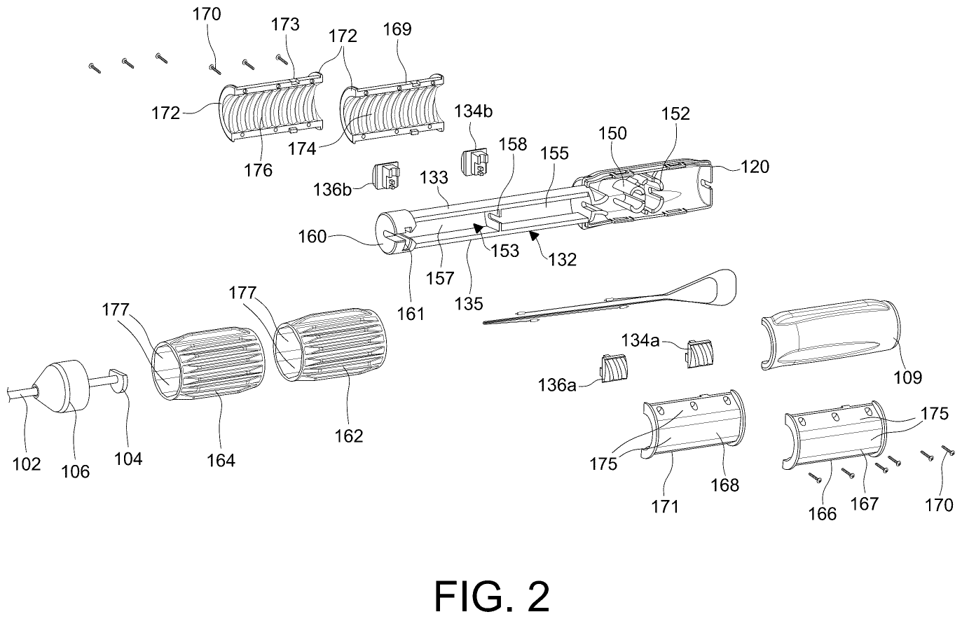

The handle 100 is coupled to the medical device 102 to enable a user to change the shape of the medical device 102 to manipulate or steer the medical device 102 in a desired direction during use. The medical device 102 includes a proximal end (shown in FIG. 2) having a flange 104 that is removably affixed within the handle 100 by a nose cap 106. A distal portion 160 of a spindle 132 includes a groove for receiving the flange 104. The nose cap 106 is configured to secure the flange 104 in the groove 161 when connected to the distal portion 160. In one embodiment, the nose cap 106 is configured to be secured to the distal portion 160 by a friction fit or snap-fit connector. In another embodiment, the nose cap 106 includes a threaded inner portion configured to interact with threads of the distal portion 160.

As illustrated in FIG. 1, the handle 100 include nose cap 106, a grip 108, a proximal knob 110, and a distal knob 112. The grip 108 includes a proximal end 120 and a grip indicator rib 122. The grip indicator rib 122 provides a reference for the medical device 102 in a neutral, or non-deflected orientation. The proximal knob 110 includes a proximal indicator rib 124 and the distal knob 112 includes a distal indicator rib 126. The proximal knob 110 and the distal knob 112 are configured to be rotated in both a clockwise and counterclockwise direction to change a shape of the medical device 102. When the indicator ribs 124, 126 are aligned with the grip indicator rib 122, the medical device 102 is substantially straight, or in a neutral position.

Rotating the proximal knob 110 in a clockwise direction puts tension on a control wire and causes the medical device 102 to deflect in a first direction. Rotating the proximal knob 110 in a counterclockwise direction puts tension on a different control wire and causes the medical device to deflect in a second direction that is substantially opposite the first direction. Similarly, rotating the distal knob 112 in a clockwise direction puts tension on another control wire and causes the medical device 102 to deflect in a third direction that is substantially orthogonal to the first and second directions. Rotating the distal knob 112 in a counterclockwise direction puts tension on an additional control wire and causes the medical device to deflect in a fourth direction that is substantially opposite the third direction. By adjusting both the proximal knob 110 and the distal knob 112 simultaneously, the medical device 102 is able to achieve deflection in any direction. By comparing the locations of the position indicators 124, 126 to the grip indicator rib 122, a user can visualize a shape of the medical device 102 located in a patient.

FIG. 2 in an exploded view of the handle assembly of FIG. 1, in accordance with an embodiment of the present disclosure. The grip 108 is formed by two halves joined together by one or more connectors 128, such as clips or snap-fit connectors. A left grip portion 109 joins with a right grip portion 111 to form the grip 108. The grip 108 is substantially hollow and includes a proximal end 120 and a distal end 130. The grip 108 houses a first pulley 150, a second pulley 152, and a guide funnel 154. A spindle 132 extends from the distal end 130 of the grip 108. The spindle 132 acts as a support for components of the handle 100.

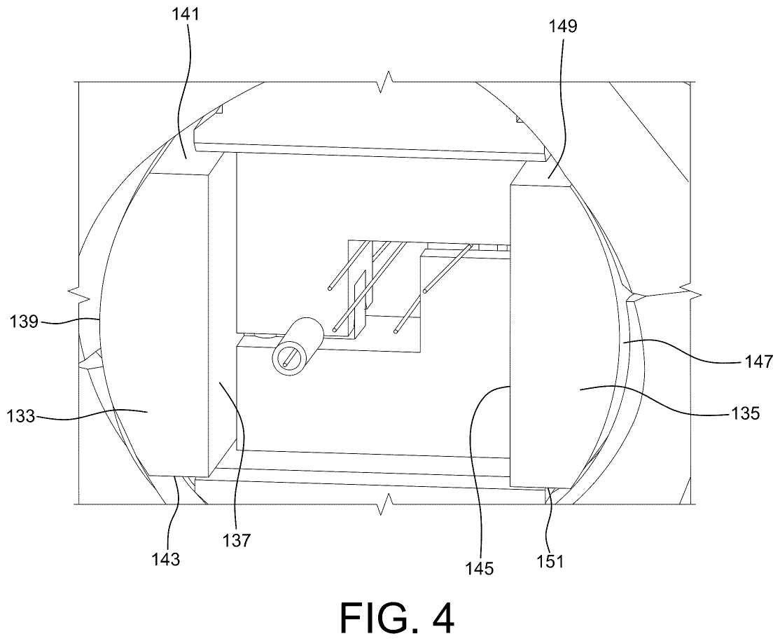

The spindle 132 includes a first wall 133 and a second wall 135 that extends parallel to the first wall 133 along a longitudinal axis of the handle 100. As illustrated in FIG. 4, the first wall 133 includes a planar inner surface 137, a curved outer surface 139, a left planar surface 141, and a right planar surface 143. The left planar surface 141 and the right planar surface 143 are orthogonal to the planar inner surface 137. Likewise, the second wall 135 includes a planar inner surface 145, a curved outer surface 147, a left planar surface 149, and a right planar surface 151. The left planar surface 149 and the right planar surface 151 are orthogonal to the planar inner surface 145. The planar inner surface 137 of the first wall 133 and the planar inner surface 145 of the second wall 135 define a guide channel 153. A divider wall 138 within the guide channel 153 separates the guide channel 153 into a proximal guide channel 155 and a distal guide channel 157.

Positioned within and translatable along the guide channel 153 are a proximal wire holder 134 and a distal wire holder 136. The proximal wire holder 134 is movable along the proximal guide channel 155 between the divider wall 138 and the distal end 130 of the grip 108. The distal wire holder 136 is movable along the distal guide channel 157 between the divider wall 138 and a distal portion 160 of the spindle 132. As discussed further below, the proximal wire holder 134 is caused to move along the proximal guide channel 155 by rotation of the proximal knob 110 and the distal wire holder 136 is caused to move along the distal guide channel 153 by rotation of the distal knob 112.

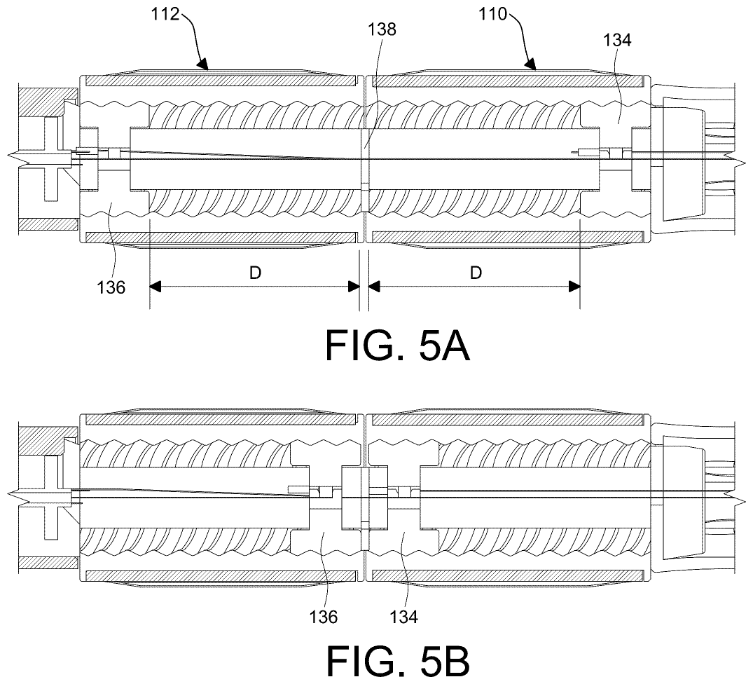

FIG. 5A is a cross-sectional view of the handle assembly along line A-A of FIG. 1 showing the distal wire holder 136 in a most distal position and the proximal wire holder 134 in a most proximal position. FIG. 5B illustrates the distal wire holder 136 in a most proximal position and the proximal wire holder 134 in a most distal position. Both the proximal wire holder 134 and the distal wire holder 134 can travel a distance D between the divider wall 138 and the distal end 130 of the grip and the distal portion 160 of the spindle 132, respectively. In some embodiments, the distance D is between 1 inch and 2 inches, for example 1.5.”

In some embodiments, the distance D is the same for both the proximal wire holder 134 and the distal wire holder 134. In other embodiments, the distance D is different for the proximal wire holder 134 and the distal wire holder 134. A longer distance D allows for greater curvature of the medical device 102 between a neutral position and a maximum deflection. A shorter distance D reduces the overall curvature of the medical device 102 between a neutral position and a maximum deflection. Varying the distance D between the proximal wire holder 134 and the distal wire holder 134 would allow for the creation of different amounts of curvature in different orientations. For example, a shorter distance D for the proximal wire holder 134 and a longer distance D for the distal wire holder 134 would allow for a smaller range of deflection in a first and second direction while allowing for a larger range of deflection in a third and fourth direction. Likewise, a longer distance D for the proximal wire holder 134 and a shorter distance D for the distal wire holder 134 would allow for a greater range of deflection in a first and second direction while allowing for a smaller range of deflection in a third and fourth direction.

The proximal knob 110 and the distal knob 112 include an outer portion surrounding a cylindrical inner portion. The proximal knob outer portion 162 and the distal knob outer portion 164 can be seen in FIG. 2. The proximal knob outer portion 162 and the distal knob outer portion 164 are formed from a flexible polymeric material capable of being stretched over the cylindrical inner portion. The polymeric material provides sufficient gripability and comfort so a user can easily rotate the knobs 110, 112 during use.

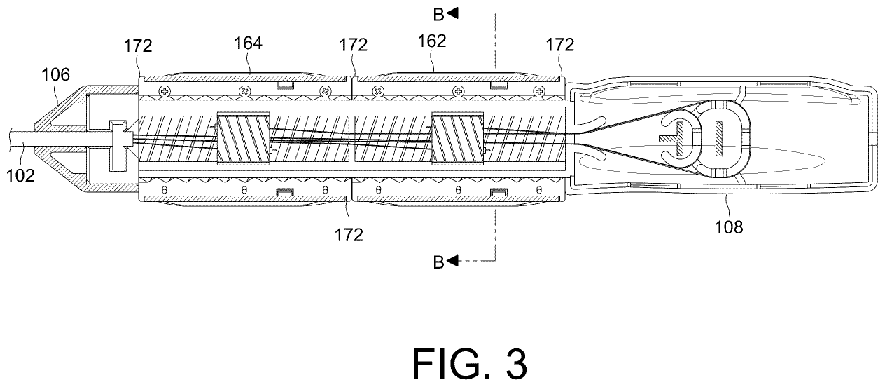

The proximal knob cylindrical inner portion 166 and the distal knob cylindrical inner portion 168 are formed from two halves joined together. The proximal knob cylindrical inner portion 166 includes a right half 169 and a left half 167 joined by a plurality of fasteners 170, for example screws. Likewise, the distal knob cylindrical inner portion 186 includes a right half 173 and a left half 171 joined by a plurality of fasteners 170. The proximal knob cylindrical inner portion 166 and the distal knob cylindrical inner portion 168 include collars 172 configured for maintaining the flexible outer portions 162, 164 in place. As can be seen in FIG. 3, the collars 172 extend above an outer surface of the inner portions 166, 168 to provide a region that inner portions 166, 168 are retained within. Adhesives may be placed on the outer surface of the inner portions 166, 168 prior to receiving the outer portions 162, 164.

The proximal knob cylindrical inner portion 166 and the distal knob cylindrical inner portion 168 each include an outer surface that includes a plurality of planar surfaces 175. The planar surfaces 175 are angled relative to one another to form a polygonal outer surface. In some embodiments, the polygonal outer surface is hexagonal. In other embodiments, the polygonal outer surface is octagonal.

The proximal knob outer portion 162 and the distal knob outer portion 164 each include an inner surface having a plurality of planar inner surfaces 177. The plurality of planar inner surfaces 177 create a polygonal inner surface that corresponds to the polygonal outer surface of the proximal knob cylindrical inner portion 166 and the distal knob cylindrical inner portion 168, respectively. This provides torque transmission and keying such that rotation of the outer portions 162, 164 results in rotation of the inner portions 166, 168 without slippage.

The proximal knob cylindrical inner portion 166 includes internal threads 174 and the distal knob cylindrical inner portion 168 includes internal threads 176. The internal threads 174, 176 can take the form of helical or screw type threads. The proximal knob internal threads 174 are configured to interact with the proximal wire holder 134 so that the proximal wire holder 134 translates along the proximal guide channel 155 upon rotation of proximal knob 110. The distal knob internal threads 176 are configured to interact with the distal wire holder 136 so that the distal wire holder 136 translates along the distal guide channel 157 upon rotation of distal knob 112. The internal threads 174, 176 are self-locking such that the knobs 110, 112 remain in place once rotated to a desired position. As such, a user merely needs to rotate the knobs 110, 112 so that the medical device 102 has a desired shape. Once the medical device 102 has the desired shape it will retain the desired shape. In some embodiments, the internal threads can include a pitch such that the distance d traveled by the wire holders 134, 136 is achieved by less than one turn of the knobs 110, 112. In some embodiments, the internal threads can include a pitch such that the distance d traveled by the wire holders 134, 136 is achieved by more than one turn of the knobs 110, 112. In some embodiments, the internal threads can include a pitch such that the distance d traveled by the wire holders 134, 136 is achieved by a single turn of the knobs 110, 112.

As shown in FIG. 2, the proximal wire holder 134 and the distal wire holder 136 are formed by two distinct pieces, a first piece 134a, 136a and a second piece 134b, 136b. During assembly, the two pieces are placed on opposite sides of the guide channel 153 and brought together to secure the control wires therebetween. As shown in FIG. 4, when located in the guide channel 153, the wire holders 134, 136 have surfaces that contact the planar inner surfaces 137,145, the left planar surfaces 141, 149, and the right planar surfaces 143, 151 of the of the first wall 133 and the second wall 135.

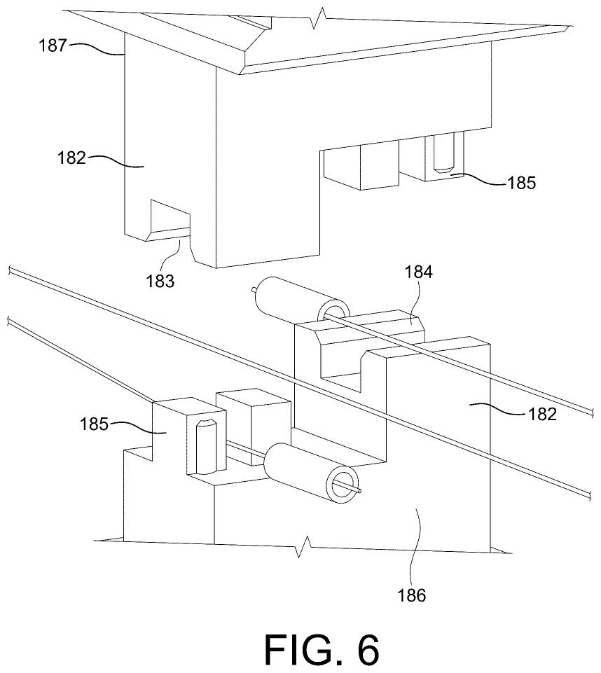

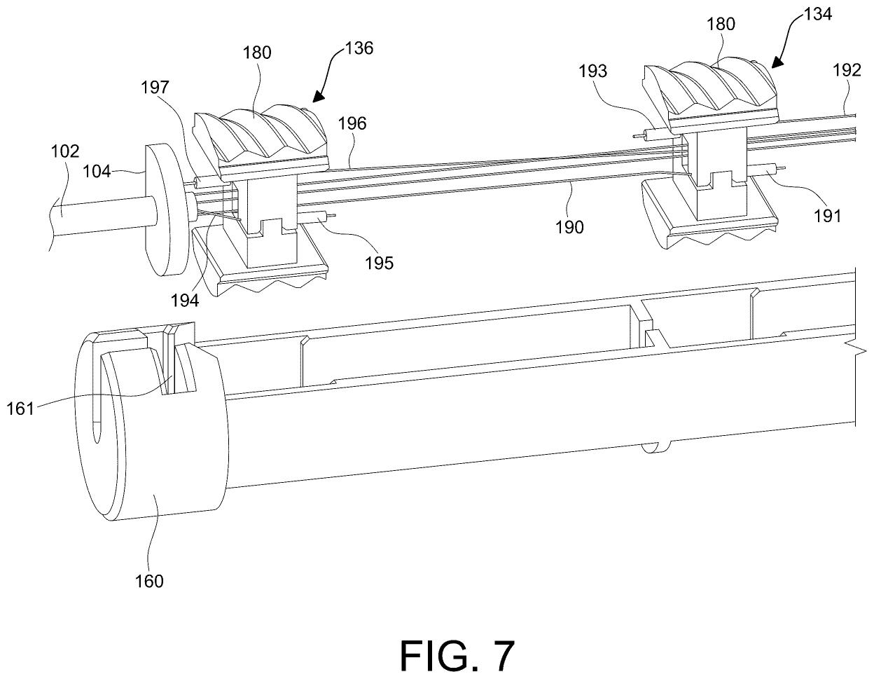

The wire holders 134, 136 are substantially identical. Referring to FIG. 7, each piece of the wire holders 134, 136 includes a curved threaded portion 180 configured to mate with the internal threads 174, 176 of the cylindrical inner portions 166, 168 of the proximal knob 110 and the distal knob 112, respectively. When assembled, the curved threaded portions 180 are opposite one another. As illustrated in FIG. 6, each piece of the wire holders 134, 136 include an extension 182 having a space 183. The space 183 is configured to receive a pair of guides 185. The guides 185 receive one of the plurality of control wires such that when the two pieces 134a, 136a, 134b, 136b are brought together, one of the control wires is trapped between the guides 185 and the extension 182. The extension 182 includes a beveled surface 184 facing the space 183 to aid in insertion of the guides 185 into the space 183. When assembled, the first piece 134a, 136a is spaced from the second piece 134b, 136b enough that the control wires are free to slide between the wire holders 134, 136 as the wire holders 134, 136 translate along the guide channel 153. Additionally, as shown in FIG. 4, an opening 188 is created that allows for some of the control wires to pass through when the two pieces 134a, 134b, 136a, 136b are joined together.

Two of the control wires are secured by the proximal wire holder 134 and the remaining two control wires are secured by the distal wire holder 136. Referring to FIG. 7, a first control wire 190 extends from the connection at the distal end of the medical device 102 into the handle 100 and through a first of the guides 185 of the proximal wire holder 134. The first control wire 190 includes a crimp 191 configured to contact a proximal surface 186 of the proximal wire holder 134 when the proximal wire holder is translated in a proximal direction along the guide channel 153. A second control wire 192 extends from the connection at the distal end of the medical device 102 into the handle 100 and through the opening 188 of the proximal wire holder 134 and into the grip 108. The second control wire 192 loops around the first pulley 150 and passes through a second set of the guides 185 of the proximal wire holder 134. The second control wire 192 includes a crimp 193 configured to contact a distal surface 187 of the proximal wire holder 134 when the proximal wire holder is translated in a distal direction along the guide channel 153.

Additionally, a third control wire 194 extends from the connection at the distal end of the medical device 102 into the handle 100 and through a first of the guides 185 of the distal wire holder 136. The third control wire 194 includes a crimp 195 configured to contact a proximal surface 186 of the distal wire holder 136 when the distal wire holder 136 is translated in a proximal direction along the guide channel 153. A fourth control wire 196 extends from the connection at the distal end of the medical device 102 into the handle 100 and through the openings 188 of the distal wire holder 136 and the proximal wire holder 134 into the grip 108. The fourth control wire 196 loops around the second pulley 152 and passes back through the opening 188 of the proximal wire holder 134 and through a second set of the guides 185 of the distal wire holder 136. The fourth control wire 196 includes a crimp 197 configured to contact a distal surface 187 of the distal wire holder 136 when the distal wire holder 136 is translated in a distal direction along the guide channel 153.

The proximal wire holder 134 is actuatable by rotation of the proximal knob 110. When the proximal knob 110 is rotated in a first direction, proximal knob internal threads 174 rotate which interact with the curved threaded portions 180 of the proximal wire holder 134 to cause the proximal wire holder 134 to translate proximally in the proximal guide channel 155. The crimp 191 of the first control wire 190 abuts a proximal surface 186 of the proximal wire holder 134 causing tensioning of the first control wire 190 while the second control wire 192 is free to slide through the proximal wire holder 134. This produces a change in a deflection of the medical device 102 in a first direction. When the proximal knob 110 is rotated in a second direction, proximal knob internal threads 174 rotate which interact with the curved threaded portions 180 of the proximal wire holder 134 to cause the proximal wire holder 134 to translate distally in the proximal guide channel 155. The crimp 193 of the second control wire 192 abuts a distal surface 187 of the proximal wire holder 134 causing tensioning of the second control wire 192 while the first control wire 190 is free to slide through the proximal wire holder 134. This produces a change in a deflection of the medical device 102 in a second direction that is substantially opposite the first direction.

Similarly, the distal wire holder 136 is actuatable by rotation of the distal knob 112. When the distal knob 112 is rotated in a first direction, distal knob internal threads 176 rotate which interact with the curved threaded portions 180 of the distal wire holder 136 to cause the distal wire holder 136 to translate proximally in the distal guide channel 153. The crimp 195 of the third control wire 194 abuts a proximal surface 186 of the distal wire holder 136 causing tensioning of the third control wire 194 while the fourth control wire 196 is free to slide through the distal wire holder 136. This produces a change in a deflection of the medical device 102 in a third direction. When the distal knob 112 is rotated in a second direction, distal knob internal threads 176 rotate which interact with the curved threaded portions 180 of the distal wire holder 136 to cause the distal wire holder 136 to translate distally in the distal guide channel 153. The crimp 197 of the fourth control wire 196 abuts a distal surface 187 of the distal wire holder 136 causing tensioning of the fourth control wire 196 while the third control wire 194 is free to slide through the distal wire holder 136. This produces a change in a deflection of the medical device 102 in a fourth direction that is substantially opposite the third direction.

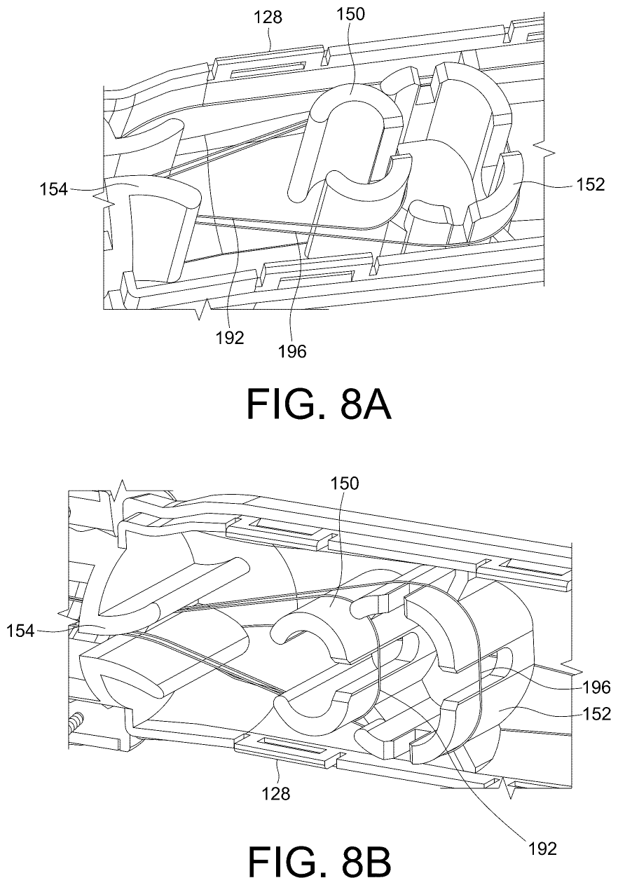

As illustrated in FIGS. 8A and 8B, the first pulley 150 is located distal of the second pulley 152. The guide funnel 154 is configured to aid in guiding the second control wire 192 and the fourth control wire 196 between the spindle 132 and the grip 108. The guide funnel 154 is located distal of the first pulley 150. As shown, the second pulley 152 has a radius that is larger than a radius of the first pulley 150. This allows for the fourth control wire 196 to loop around the first pulley 150 and not interfere with the second control wire 192. The first pulley 150 and the second pulley 152 are configured to allow for the second control wire 192 and the fourth control wire 196 to change direction. The first pulley 150 and the second pulley 152 can take the form of one or more rotating member, post, or fixed curved surfaces.

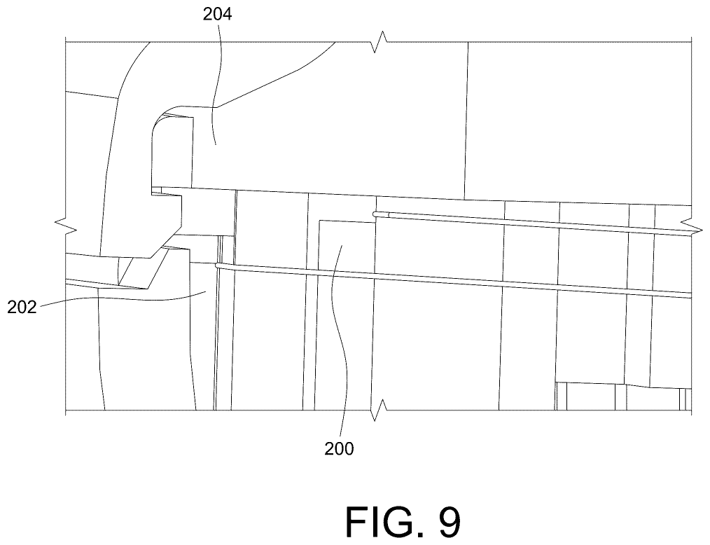

In order to ensure the second control wire 192 and the fourth control wire 196 remain on the first pulley 150 and the second pulley 152, respectively, the left grip portion 109 and the right grip portion 111 include a plurality of ribs that extend from an inner surface of the grip 108. As shown in FIG. 9, the left grip portion 109 includes a first rib 200 that supports the second control wire 192 and a second rib 202 that supports the fourth control wire 196. Second control wire 192 and fourth control wire 196 rest on ribs 200, 202 and are secured in place by a rib 204 extending from an internal surface of the right grip portion 111.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A handle for a steerable medical device, the medical device including a plurality of control wires, a distal end of the plurality of control wires being coupled to the medical device at a distal region thereof, the handle comprising:
a grip having a distal end and a proximal end;
a spindle extending from the distal end of the grip, the spindle including a first wall and a second wall parallel to the first wall, a planar inner surface of the first wall and the second wall defining a guide channel;
a proximal wire holder translatable along the guide channel;
a distal wire holder translatable along the guide channel;
a proximal knob rotatably coupled to the spindle for moving the proximal wire holder in a proximal direction to manipulate a first control wire of the plurality of control wires to produce a change in a deflection of the medical device in a first direction, and for moving the proximal wire holder in a distal direction to manipulate a second control wire of the plurality of control wires to produce a change in a deflection of the medical device in a second direction opposite the first direction; and
a distal knob rotatably coupled to the spindle for moving the distal wire holder in a proximal direction to manipulate a third control wire of the plurality of control wires to produce a change in a deflection of the medical device in a third direction and for moving the distal wire holder in a distal direction to manipulate a fourth control wire of the plurality of control wires to produce a change in a deflection of the medical device in a fourth direction opposite the third direction.

2. The handle of claim 1, further comprising a nose cap, wherein the spindle includes a distal end having a groove for receiving a flange from a medical device and the nose cap is configured to secure the flange in the groove.

3. The handle of claim 1, wherein the first wall and the second wall each include a curved outer surface, and a left and right planar surface orthogonal to the planar inner surface.

4. The handle of claim 1, wherein the spindle comprises a divider wall within the guide channel separating the guide channel into a proximal guide channel and a distal guide channel, and the proximal wire holder is limited to movement within the proximal guide channel and the distal wire holder is limited to movement within the distal guide channel.

5. The handle of claims 1, wherein the grip is hollow and comprises a right half and a left half connected by one or more connector.

6. The handle of claim 5, wherein the grip houses a first pulley, second pulley, and a guide funnel.

7. The handle of claim 6, wherein the first pulley is located distal of the second pulley, the guide funnel is located distal of the first pulley, and the second pulley has a radius larger than a radius of the first pulley.

8. The handle of claim 1, wherein the proximal knob and the distal knob include an outer portion surrounding a cylindrical inner portion, the cylindrical inner portion including internal threads.

9. The handle of claim 8, wherein the inner portion comprises a right half and a left half.

10. The handle of claim 8, wherein the proximal wire holder includes curved threaded portions configured to mate with the internal threads of the cylindrical inner portion of the proximal knob, and the distal wire holder includes curved threaded portions configured to mate with the internal threads of the cylindrical inner portion of the distal knob.

11. The handle of claim 1, wherein the proximal wire holder comprises a first piece and a second piece, the first piece and the second piece being configured to join together to create an opening and to secure the first control wire in a first notch and the second control wire in a second notch, the first control wire including a crimp configured to contact a proximal surface of the proximal wire holder when the proximal wire holder is translated in a proximal direction along the guide channel, and the second control wire including a crimp configured to contact a distal surface of the proximal wire holder when the proximal wire holder is translated in a distal direction along the guide channel.

12. The handle of claim 11, wherein the distal wire holder comprises a third piece and a fourth piece, the third piece and the fourth piece being configured to join together to create an opening and to secure the third control wire in a third notch and the fourth control wire in a fourth notch, the third control wire including a crimp configured to contact a proximal surface of the distal wire holder when the distal wire holder is translated in a proximal direction along the guide channel, and the fourth control wire including a crimp configured to contact a distal surface of the distal wire holder when the distal wire holder is translated in a distal direction along the guide channel.

13. The handle of claim 1, wherein the proximal knob includes a proximal indicator rib, the distal knob includes a distal indicator rib, and the grip includes a grip indicator rib, such that alignment of the proximal indicator rib, the distal indicator rib, and the grip indicator rib indicates a neutral position of the medical device.

14. A handle for a steerable medical device, the medical device including a plurality of control wires, a distal end of the plurality of control wires being coupled to the medical device at a distal region thereof, the handle comprising:
a grip having a distal end and a proximal end;
a spindle extending from the distal end of the grip, the spindle including a first wall and a second wall parallel to the first wall, a planar inner surface of the first wall and the second wall defining a guide channel; and
a proximal wire holder translatable along the guide channel, the proximal wire holder including a first piece and a second piece, the first piece and the second piece having curved threaded portions, the first piece and the second piece being configured to join together to create an opening and to secure a first control wire of the plurality of control wires in a first notch and the second control wire of the plurality of control wires in a second notch, the first control wire including a crimp configured to contact a proximal surface of the proximal wire holder when the proximal wire holder is translated in a proximal direction along the guide channel, and the second control wire including a crimp configured to contact a distal surface of the proximal wire holder when the proximal wire holder is translated in a distal direction along the guide channel.

15. The handle of claim 14, further comprising a proximal knob rotatably coupled to the spindle for moving the proximal wire holder in the proximal direction to manipulate the first control wire to produce a change in a deflection of the medical device in a first direction, and for moving the proximal wire holder in the distal direction to manipulate the second control wire to produce a change in a deflection of the medical device in a second direction opposite the first direction.

16. A handle for a steerable medical device, the medical device including a plurality of control wires, a distal end of the plurality of control wires being coupled to the medical device at a distal region thereof, the handle comprising:
a grip having a distal end and a proximal end;
a spindle extending from the distal end of the grip, the spindle including a first wall and a second wall parallel to the first wall, a planar inner surface of the first wall and the second wall defining a guide channel, a divider wall within the guide channel separating the guide channel into a proximal guide channel and a distal guide channel;
a proximal wire holder translatable along the proximal guide channel; and
a proximal knob rotatably coupled to the spindle for moving the proximal wire holder in a proximal direction to manipulate a first control wire of the plurality of control wires to produce a change in a deflection of the medical device in a first direction, and for moving the proximal wire holder in a distal direction to manipulate a second control wire of the plurality of control wires to produce a change in a deflection of the medical device in a second direction opposite the first direction.

17. The handle of claim 16, further comprising:
a distal wire holder translatable along the distal guide channel; and
a distal knob rotatably coupled to the spindle for moving the distal wire holder in a proximal direction to manipulate a third control wire of the plurality of control wires to produce a change in a deflection of the medical device in a third direction and for moving the distal wire holder in a distal direction to manipulate a fourth control wire of the plurality of control wires to produce a change in a deflection of the medical device in a fourth direction opposite the third direction.

18. The handle of claim 16, further comprising a nose cap, wherein the spindle includes a distal end having a groove for receiving a flange from a medical device and the nose cap is configured to secure the flange in the groove.

19. The handle of claim 17, wherein the proximal knob and the distal knob include an outer portion surrounding a cylindrical inner portion, the cylindrical inner portion including internal threads.

20. The handle of claim 18, wherein the proximal wire holder includes curved threaded portions configured to mate with the internal threads of the cylindrical inner portion of the proximal knob, and the distal wire holder includes curved threaded portions configured to mate with the internal threads of the cylindrical inner portion of the distal knob.
